(19) United States
(12) Patent Application Publication
(10) Pub. No.: US 2025/0339366 A1
(43) Pub. Date: Nov. 6, 2025

(54) AN ORODISPERSIBLE TABLET OF RIVAROXABAN

(71) Applicant: INTAS PHARMACEUTICALS LTD., Ahmedabad, Gujarat (IN)

(72) Inventors: Noé GUZMÁN DEL CASTILLO, Sevilla (ES); María Teresa MÁRQUEZ QUIDIELLO, Sevilla (ES); Angel José MUÑOZ RUIZ, Sevilla (ES); María Carmen LLUCH LORES, Barcelona (ES); Xavier FORMOSA MÁRQUEZ, Barcelona (ES)

(21) Appl. No.: 18/854,146

(22) PCT Filed: Apr. 4, 2023
(86) PCT No.: PCT/IB2023/053383

§ 371 Date: Oct. 4, 2024

(30) Foreign Application Priority Data
Apr. 8, 2022 (IN) 202221021179

(51) Int. Cl. A61K 9/00; A61K 9/16; A61K 9/20; A61K 31/5377

(52) U.S. Cl. CPC A61K 9/0056; A61K 9/1688; A61K 9/2018; A61K 9/2027; A61K 9/2054; A61K 9/2059; A61K 31/5377

(57) ABSTRACT

The present invention relates to an orodispersible tablet of rivaroxaban having overall improved characteristics. its process of manufacturing and its use as anticoagulant.

### RELATED APPLICATIONS

This application is related to Indian Provisional Application No. IN202221021179 filed on 08^{th} April 2022 and is incorporated herein in its entirety.

### FIELD OF THE INVENTION

The present invention relates to an orodispersible tablet of rivaroxaban having overall improved characteristics, its process of manufacturing and its use as anticoagulant.

### BACKGROUND OF THE INVENTION

Rivaroxaban is represented by the structural formula (I) and its chemical name is 5-Chloro-N-({(5S)-2-oxo-3-[4-(3-oxo-4-morpholinyl)phenyl]-1,3-oxzolidin-5-yl}methyl)-2-thiophene-carboxamide.

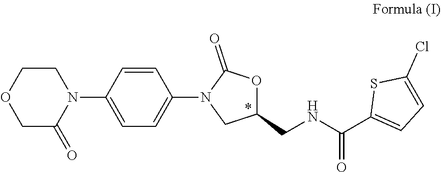 [SMILES: O=C(NC[C@H]1CN(c2ccc(N3CCOCC3=O)cc2)C(=O)O1)c1ccc(Cl)s1]

In Europe, rivaroxaban (Xarelto®) was approved by the European Medicines Agency (EMA) in September 2008, whereas in the US, the Food and Drug Administration (FDA) approved rivaroxaban (Xarelto®) in July 2011. Xarelto® was approved as well by the Japanese Pharmaceuticals and Medical Devices Agency (PMDA) in January 2012. Rivaroxaban is indicated in EU, US and Japan in the treatment of: a) prevention of venous thromboembolism (VTE) in adult patients undergoing elective hip or knee replacement surgery; and/or b) treatment of deep vein thrombosis (DVT) and pulmonary embolism (PE), and prevention of recurrent DVT and PE in adults; and/or c) prevention of stroke and systemic embolism in adult patients with non-valvular atrial fibrillation with one or more risk factors, such as congestive heart failure, hypertension, age ≥75 years, diabetes mellitus, prior stroke or transient ischaemic attack; and/or d) treatment of venous thromboembolism (VTE) and prevention of VTE recurrence in children and adolescents aged less than 18 years and weighing either from 30 kg to 50 kg or more than 50 kg after at least 5 days of initial parenteral anticoagulation treatment; and/or e) treatment of venous thromboembolism (VTE) and prevention of VTE recurrence in term neonates, infants and toddlers, children, and adolescents aged less than 18 years after at least 5 days of initial parenteral anticoagulation treatment; and/or f) inhibition of ischemic stroke and systemic embolism in patients with non-valvular atrial fibrillation; and/or g) treatment and suppression of recurrence of venous thromboembolism; and/or h) co-administered with acetylsalicylic acid (ASA) alone or with ASA plus clopidogrel or ticlopidine, prevention of atherothrombotic events in adult patients after an acute coronary syndrome (ACS) with elevated cardiac biomarkers; and/or i) co-administered with acetylsalicylic acid (ASA), prevention of atherothrombotic events in adult patients with coronary artery disease (CAD) or symptomatic peripheral artery disease (PAD) at high risk of ischaemic events.

Rivaroxaban (Xarelto®) is commercially available in Europe in the dosage form of immediate-release film-coated tablets and as granules for oral suspension. Both dosage forms need to be swallowed—meaning that patients in therapy should be able to perform the action of swallowing correctly. However, swallowing may prove difficult for some category of patients, for example, for elderly patients or pediatric patients, or for patients who barely cooperate with medical personnel because of progression of disabling diseases. In these clinical situations the patient has difficulty in swallowing, and therefore it would be advisable to replace the tablets or the granules for oral suspension that are to be swallowed with water with other oral dosages forms in which swallowing is made easier.

A particular solid pharmaceutical dosage form that rapidly disintegrates are orodispersible tablets (also named orally disintegrated tablet (ODT) or fast disintegrating tablets), which do not require water, and hence can be consumed in situations where patients require an easy-to-administer dosage form. Orodispersible tablets are an improved dosage form with respect to conventional tablets, as they are not required to be swallowed and are in line with the current fast-paced modern lifestyle. Furthermore, orodispersible tablets are especially suitable for patients suffering from dysphagia, wherein closure of the glottis and simultaneous contraction of the muscles of the larynx do not propel adequately the conventional tablet that needs to be swallowed.

ODT's are distinguished from conventional sublingual tablets, buccal tablets and lozenges by their fast disintegration time in the oral cavity, which generally takes around 60 seconds or less. In fact, the FDA Center for Drug Evaluation and Research (CDER) defines orodispersible tablets as: “A solid dosage form containing medicinal substances which disintegrates rapidly, usually within a matter of seconds, when placed upon the tongue”. Therefore, it is commonly accepted in the art of developing/manufacturing orodispersible tablets that such tablets, when placed in the mouth, should disintegrate in less than one minute—otherwise, if disintegration takes longer, the attractiveness of orodispersible tablets is lost, as the taste buds of the patients' tongue and other receptors of the oral cavity are extensively exposed to a direct contact with the active ingredient. Moreover, for those patients who are travelling, or which have limited access to water, it may be discomfortable to take an orodispersible tablet which does not disintegrate rapidly (i.e. in less than one minute).

Rivaroxaban is practically insoluble in water and has high permeability, thus being a BCS class II compound. Due to low solubility of rivaroxaban, the use of particles with small particle size distribution (PSD) is required in order to enhance solubility and therefore obtaining bioavailable rivaroxaban compositions. However, it is known in the art that such small particle size distribution of rivaroxaban adversely affects the content uniformity of the finished medicinal products, as well as it has a detrimental effect in blend flowability during manufacturing process. Furthermore, a small particle size distribution causes an undesirable effect of segregation of the active ingredient with respect to other pharmaceutically acceptable excipients of the product along the manufacturing process. The most employed strategy in the prior art for overcoming those problems has been to produce pharmaceutical dosage forms (i.e. film-coated tablets) containing rivaroxaban by moist granulation (i.e. wet-granulation). By producing granules, adequate content uniformity is achieved in the finished product, whereas acceptable flowability levels are obtained. For example, WO05060940 discloses rivaroxaban film coated tablets using granules comprising rivaroxaban in hydrophilized form that are produced by moist granulation.

Specifically in relation to orodispersible tablets, there is a single disclosure in the prior art directed to orodispersible tablet comprising rivaroxaban. JP2021195335 discloses a method for producing an orally disintegrating tablet of rivaroxaban by wet-granulation method. Therefore, following the established practice in the field of rivaroxaban medicinal compositions, JP2021195335 teaches to the person skilled in the art that a granulation process is indispensable for obtaining rivaroxaban orodispersible tablets having acceptable characteristics. Disclosed examples therein are manufactured by preparing rivaroxaban containing granules, followed by further mixing with a water-soluble polymer excipient and final tabletting.

However, JP2021195335 suffers from the serious drawback of being manufactured by wet-granulation processes which require several processing steps and long manufacturing times, thus making the overall process lengthy and with high cost. Moreover, orodispersible rivaroxaban tablets made by wet-granulation techniques tend to deliver finished drug formulations with variability with respect to the dissolution rate of rivaroxaban. Another limitation of the known orodispersible rivaroxaban tablet is their stability, as during wet-granulation steps rivaroxaban is subjected to humidity conditions (water or other solvents) or to high temperatures required for drying obtained rivaroxaban granules. Such harsh conditions result in stability problems of the manufactured orodispersible rivaroxaban tablets.

When developing new orodispersible tablets, it should be borne in mind that it is a standard requirement by regulatory agencies to prove strict bioequivalence criteria of said newly developed orodispersible tablets versus the conventional film coated tablet of the reference product. For instance, in Europe, according to the Guideline for Bioequivalence (CPMP/EWP/QWP/1401/98 Rev.1/Corr., 2010), if it cannot be demonstrated that the active ingredient is not absorbed in the oral cavity, bioequivalence for new developed orodispersible tablets must be evaluated in human studies. Therefore, said requirement of bioequivalence poses considerable challenges for the development of new orodispersible tablets.

Therefore, in view of the prior art, there is the need to provide for rivaroxaban orodispersible tablets having overall improved characteristics. In this regard, there is room for improvement in the prior art for providing rivaroxaban orodispersible tablets obtainable by a fast, straightforward and low-cost process while having at the same time improved disintegration time, stability, content of uniformity, friability and hardness. Furthermore, there is the need to provide for rivaroxaban tablets which, from a regulatory standpoint, exhibit reliable and adequate dissolution profile of rivaroxaban for resulting in being bioequivalent to Xarelto® immediate-release film-coated tablets.

### SUMMARY OF THE INVENTION

The inventors of the present invention have been capable of designing new orodispersible tablets comprising rivaroxaban particles having a D(v, 90) less than or equal to 20.00 μm, a pharmaceutically acceptable disintegrant, and one or more further pharmaceutically acceptable excipients, wherein said orodispersible tablet is obtainable by a direct compression process of a dry powdered mixture comprising said rivaroxaban particles, the pharmaceutically acceptable disintegrant, and the one or more further pharmaceutically acceptable excipients.

By manufacturing orodispersible tablets of the invention by a direct compression process, the manufacture of a complete batch of rivaroxaban tablets is made within few hours, thus allowing complete production in a single-day shift at the manufacturing site. This represents an improvement with respect to wet-granulation manufacturing processes known in the art (JP2021195335), which require more than the standard 8-hours shift in a manufacturing site. In other words, the time employed in the manufacturing of a complete batch of rivaroxaban orodispersible tablets according to the invention (i.e. by a direct compression process) is less than half as the one required for manufacturing of a complete batch of rivaroxaban orodispersible tablets according to JP2021195335. Thus, direct compression process of the invention provides an improvement in time of manufacturing, leading to a reduction of in economic and energetic manufacturing costs.

Even more, the inventors have surprisingly found that orodispersible tablets of the invention, obtained by direct compression, comprising rivaroxaban particles having a D(v, 90) less than or equal to 20.00 μm, a pharmaceutically acceptable disintegrant, and one or more further pharmaceutically acceptable excipients, have excellent content uniformity as well as overall improved technical characteristics. In other words, inventors have unexpectedly found that despite of using a small particle size distribution (PSD) of rivaroxaban particles, and contrary to the common established practice in the field by which when using rivaroxaban particles having a small PSD said rivaroxaban must be granulated, a direct compression method (i.e. without granulation steps) allows to obtain rivaroxaban containing orodispersible tablets with excellent values of disintegrability, stability, content of uniformity, friability and hardness. In particular, the inventors have found orodispersible tablets according to the invention, obtained by direct compression, comprising rivaroxaban particles having a D(v, 90) less than or equal to 20.00 μm, a pharmaceutically acceptable disintegrant, and one or more further pharmaceutically acceptable excipients, indeed satisfactorily disintegrate rapidly, in particular in less than two minutes, particularly in less than one minute and a half, more particularly in less than one minute, and even more particularly in less than 55 seconds. In particular, orodispersible tablets of the invention disintegrate in the time range of from two minutes and 5 seconds, preferably in the time range of from one minute and 40 seconds and 10 seconds, more preferably in the time range of from one minute and 30 seconds and 15 seconds, and even more preferably in the time range of from one minute and 15 seconds and 20 seconds.

Rivaroxaban orodispersible tablets of the invention have adequate hardness and low friability, which are important advantages in order to withstand physical shocks along manufacturing process and for storage and handling transportation. Moreover, rivaroxaban orodispersible tablets of the invention have an improved stability with respect to commercially available Xarelto® film-coated tablets. By manufacturing the orodispersible tablets of the invention via direct compression process formation of degradation products can surprisingly be prevented over time. Particularly, rivaroxaban orodispersible tablets of the invention comply with the strict criteria of impurities limit specifications required by the European or Japanese regulatory agencies.

Furthermore, rivaroxaban orodispersible tablets of the invention allow to provide reliable and adequate dissolution profile of rivaroxaban, thus releasing rivaroxaban in a sufficiently equivalent manner for being bioequivalent to immediate-release film-coated tablets of Xarelto®. By having such adequate dissolution profile, and thus being sufficiently equivalent to currently marketed Xarelto® conventional tablets, rivaroxaban orodispersible tablets of the invention avoid the risk of being either suprabioequivalent or infrabioequivalent, which may affect the toxicity and/or the efficacy of said orodispersible tablet. Therefore, orodispersible tablets of the invention constitute a valuable therapeutic tool in the field of anticoagulant medicinal products.

Advantageously, the inventors have also unexpectedly found that the presence of two different pharmaceutically acceptable disintegrants in the orodispersible tablets of the invention provide a synergistic effect in terms of disintegration time, as well as in terms of hardness and friability. In other words, the interaction of two different pharmaceutically acceptable disintegrants in the orodispersible tablets of the invention achieve an improved disintegration time, hardness and friability while providing an adequate dissolution profile of rivaroxaban.

Thus, a first aspect of the present invention relates to an orodispersible tablet comprising rivaroxaban particles having a D(v, 90) less than or equal to 20.00 μm, a pharmaceutically acceptable disintegrant, and one or more further pharmaceutically acceptable excipients, wherein said orodispersible tablet is obtainable by a direct compression process of a dry powdered mixture comprising said rivaroxaban particles, the pharmaceutically acceptable disintegrant, and the one or more further pharmaceutically acceptable excipients.

A second aspect of the invention relates to a direct compression process for the preparation of the orodispersible tablet of the first aspect of the invention, which comprises the steps of: (i) mixing rivaroxaban particles with the pharmaceutically acceptable disintegrant, and the one or more further pharmaceutically acceptable excipients; (ii) blending the mixture of step (i); (iii) adding one or more lubricants to the mixture of step (ii); (iv) blending the mixture of step (iii); (v) compressing the dry powdered mixture of step (iv) to form the orodispersible tablet.

A third aspect of the invention relates to the orodispersible tablet of the first aspect of the invention for use in: a) prevention of venous thromboembolism (VTE) in adult patients undergoing elective hip or knee replacement surgery; and/or b) treatment of deep vein thrombosis (DVT) and pulmonary embolism (PE), and prevention of recurrent DVT and PE in adults; and/or c) prevention of stroke and systemic embolism in adult patients with non-valvular atrial fibrillation with one or more risk factors, such as congestive heart failure, hypertension, age ≥75 years, diabetes mellitus, prior stroke or transient ischaemic attack; and/or d) treatment of venous thromboembolism (VTE) and prevention of VTE recurrence in children and adolescents aged less than 18 years and weighing either from 30 kg to 50 kg or more than 50 kg after at least 5 days of initial parenteral anticoagulation treatment; and/or e) treatment of venous thromboembolism (VTE) and prevention of VTE recurrence in term neonates, infants and toddlers, children, and adolescents aged less than 18 years after at least 5 days of initial parenteral anticoagulation treatment; and/or f) inhibition of ischemic stroke and systemic embolism in patients with non-valvular atrial fibrillation; and/or g) treatment and suppression of recurrence of venous thromboembolism; and/or h) co-administered with acetylsalicylic acid (ASA) alone or with ASA plus clopidogrel or ticlopidine, prevention of atherothrombotic events in adult patients after an acute coronary syndrome (ACS) with elevated cardiac biomarkers; and/or i) co-administered with acetylsalicylic acid (ASA), prevention of atherothrombotic events in adult patients with coronary artery disease (CAD) or symptomatic peripheral artery disease (PAD) at high risk of ischaemic events.

### DETAILED DESCRIPTION

The term “active ingredient” or “API” as used herein refers to a pharmaceutically active molecule (e.g. rivaroxaban) as well as its pharmaceutically acceptable and therapeutically active salts, hydrates, esters, amides, prodrugs, metabolites, enantiomers, polymorphs, analogs, etc. that induce a desired pharmacological or physiological effect. Terms like “active”, “active agent”, “active pharmaceutical ingredient”, “active substance”, “drug substance”, “active drug” may be used synonymously for “active ingredient”.

The term “rivaroxaban” as used herein corresponds to the International Non-proprietary Name (INN) for 5-Chloro-N-({(5S)-2-oxo-3-[4-(3-oxo-4-morpholinyl)phenyl]-1,3-oxzolidin-5-yl}methyl)-2-thiophene-carboxamide but also as their pharmaceutically acceptable salts and/or hydrates.

The term “rivaroxaban particles having a D(v, x),” as used herein is defined as meaning that X % of the volume of the rivaroxaban particles have a diameter equal to or less than a specified diameter. For example, the term “rivaroxaban particles having a D (v, 90) less than or equal to 20.00 μm” means that 90% of the volume of particles of rivaroxaban have a diameter less than or equal to 20.00 μm. The particle size distributions (PSD) of rivaroxaban particles referred herein refer to particle size distributions determined using techniques available in the art, such as laser light scattering technique (e.g. by using a Malvern apparatus). Alternatively, the person skilled in the art may use other equivalent apparatus for measuring particle size distribution (PSD) of rivaroxaban particles.

The term “orodispersible tablet” as used herein is defined in accordance with the European Pharmacopeia, edition 10.0, page 939, as an uncoated tablet intended to be placed in the mouth where it disperses rapidly before being swallowed, more precisely orodispersible tablets disintegrate within three minutes in the disintegration test. In accordance with this definition the term orodispersible tablet is intended to be a synonym of solid oral dosage forms named as orodispersable tablet, orodisperse tablet, orally disintegrating tablet, orally disintegrated tablet, fast disintegrating tablet, fast dissolving tablet, mouth-dissolving tablets, amongst others.

The term “disintegration test” as used herein is defined in accordance with Test A of the European Pharmacopeia (edition 10.0, page 323), water having pH=7, at 37° C. and 30 cycles per minute. The term “complete disintegration” as used herein is defined in accordance with the European Pharmacopeia, edition 10.0, page 323. Disintegration, as defined herein, does not imply complete dissolution of the dosage form or even of its active pharmaceutical ingredient.

The term “disintegrate” as used herein is defined as the action whereby a solid dosage form is brought from a solid state to a state of complete disintegration.

The term “dissolution profile” as used herein refers to dissolution over time of rivaroxaban from the orodispersible tablet of the invention. Hereinafter, the dissolution profile is measured in weight of dissolved rivaroxaban per initial weight of rivaroxaban in the orodispersible tablet, and it is expressed in weight percentage (% w/w). Unless otherwise stated, hereinafter, the dissolution profiles have been measured under pH=4.5, using Apparatus II (paddle), placing the orodispersible tablet in 900 mL at 37° C., stirring at 50 revolutions per minute and N=6. Alternatively, dissolution profiles may be measured under pH=4.5 with the presence of 0.4% sodium lauryl sulfate, using Apparatus II (paddle), placing the orodispersible tablet in 900 mL at 37° C., stirring at 75 revolutions per minute and N=6. In any case, the qualitatively gist of the dissolution profile remains the same either with the first or the alternative measurement method.

The term “friability” as used herein refers to the tendency for a tablet to chip, crumble or break during handling. The test of friability is carried out following the guidelines of the European Pharmacopeia, edition 10.0, pp. 336-337. A maximum loss of mass not greater than 1.0% is considered acceptable for most products.

The term “pharmaceutically acceptable excipient” (also named as “excipients”) refers to a substance formulated alongside with the active pharmaceutical ingredient of a medicinal product and includes all kind of pharmaceutically acceptable compounds commonly used in pharmaceutical compositions and in particular orodispersible tablets. The term “pharmaceutically acceptable excipients” comprise diluents, binders, disintegrants, surfactants, lubricants, sweeteners, glidants, colorants and flavor agents and mixtures thereof.

The term “diluent” as used herein is defined as a pharmaceutical acceptable excipient that is used as diluent in pharmaceutical compositions. The term “diluent” comprises one or combinations of two or more selected from the group of mannitol, maltol, sorbitol, maltitol, xylitol, isomalt, erythritol, lactose, starch and its derivatives such as pregelatinized starch, cellulose and its derivatives, in particular microcrystalline cellulose, and calcium phosphate. The most preferred are mannitol and microcrystalline cellulose.

The term “binder” as used herein is defined as a pharmaceutical acceptable excipient that hold the ingredients together in pharmaceutical compositions. Binders are the agents used to increase the cohesion of the powdery particles or granules during the compression, in order to obtain pharmaceutical forms with a defined hardness. Binders ensure that tablets and granules can be formed with required mechanical strength.

However, in the context of the present invention, binders are defined exclusively as agents used to increase the cohesion of the powdery particles. In other words, taking into account that orodispersible tablets of the invention are only obtainable by a direct compression process (no granulation process is involved at all), the term “binder” does not encompass agents used to prepare binder solutions for manufacturing granules.

Additionally, in the context of the present invention, the term “binder” shall not be understood as a “coating agent”, as a coating agent serves the purpose of coating the pharmaceutical dosage form (e.g. tablets) whereas binders as used herein in the present invention are pharmaceutical acceptable excipients that hold powdery particles together in pharmaceutical compositions. In other words, the term “binder” in the context of the present invention do not encompass agents which are used as coating agents or in a coating process.

The term “binder” in the context of the present invention comprises one or combinations of two or more selected from the group of hydroxypropyl methyl cellulose, hydroxypropyl cellulose, hydroxyethyl methyl cellulose, hydroxyethyl cellulose, methyl cellulose, ethyl cellulose, sodium carboxymethyl cellulose and mixtures thereof. The most preferred are hydroxypropyl methyl cellulose and hydroxypropyl cellulose.

The term “disintegrant” as used herein is defined as a pharmaceutical acceptable excipient that is used as disintegrant in pharmaceutical compositions. The term “disintegrant” comprises one or combinations of two or more selected from the group of: crospovidone, croscarmellose sodium, carmellose calcium, carmellose, calcium silicate and sodium starch glycolate and mixtures thereof.

The term “surfactant” as used herein is defined as a pharmaceutical acceptable excipient that is used as surfactant in pharmaceutical compositions. The term “surfactant” comprises one or combinations of two or more selected from the group of: sodium lauryl sulfate, polyethylene glycol sorbitan monolaurate, polyethylene glycol sorbitan monopalmitate, polyethylene glycol sorbitan monostearate, polyethylene glycol sorbitan monooleate, polaxamer, sodium dioctyl sulphosuccinate, glycerol monostearate, sorbitan monolaurate.

The term “lubricant” as used herein is defined as a pharmaceutical acceptable excipient that is used as lubricant in pharmaceutical compositions. The term “lubricant” comprises one or combinations of two or more selected from the group of talc, sodium benzoate, sodium stearyl fumarate, calcium stearate, magnesium stearate, zinc stearate, glyceryl behenate, stearic acid and glyceryl monostearate; more particularly, such lubricant is selected from the group of sodium stearyl fumarate and magnesium stearate.

The term “sweetener” as used herein is defined as a pharmaceutical acceptable excipient that is used as sweetener in pharmaceutical compositions. The term “sweetener” comprises one or combinations of two or more selected from the group of aspartame, potassium acesulfame (Acesulfame K), sodium saccharinate, neohesperidine dihydrochalcone, sucralose, sucrose, fructose, monoammonium glycyrrhizinate and mixtures thereof.

The term “glidant” as used herein is defined as a pharmaceutical acceptable excipient that is used as glidant in pharmaceutical compositions. The term “glidant” comprises one or combinations of two or more selected from the group of colloidal silicon dioxide, hydrophobic colloidal silica, magnesium oxide, magnesium silicate, magnesium trisilicate and mixtures thereof.

The term “colorant” as used herein is defined as a pharmaceutical acceptable excipient that is used as colorant in pharmaceutical compositions. The term “colorant” comprises one or combinations of two or more selected from the group of Food Yellow No.5, Food Red No. 2, and Food Blue No.2; food lake dyes, Yellow Ferric Oxide, iron sesquioxide, titanium oxide, β-carotene, riboflavin and mixtures thereof; more particularly, such colorant is selected from the group of Yellow Ferric Oxide, iron sesquioxide and titanium oxide.

The term “flavouring agent” as used herein is defined as a pharmaceutical acceptable excipient that is used as flavouring agent in pharmaceutical compositions. The term “flavouring agent” comprises one or combinations of two or more selected from the group of cherry, raspberry, apricot, pear, strawberry, bitter masker, pineapple, lemon, honey, mint garden, orange, peppermint, menthol, black currant, banana, red fruits, wild berries and caramel flavour; more particularly, such flavouring agent is selected from the group of cherry, apricot, mint and honey flavour.

The term “direct compression process of a dry powdered mixture” as used herein is defined as a process in which no solvent is used during the blending of the active ingredient (i.e. rivaroxaban) and the pharmaceutically acceptable excipients of the orodispersible tablet. Direct compression is a dry process used in the manufacturing of orodispersible tablets.

As it is mentioned above, an aspect of the present invention relates to an orodispersible tablet comprising rivaroxaban particles having a D(v, 90) less than or equal to 20.00 μm, a pharmaceutically acceptable disintegrant, and one or more further pharmaceutically acceptable excipients, wherein said orodispersible tablet is obtainable by a direct compression process of a dry powdered mixture comprising said rivaroxaban particles, the pharmaceutically acceptable disintegrant, and the one or more further pharmaceutically acceptable excipients.

The orodispersible tablets of the invention may further comprise a second pharmaceutically acceptable disintegrant which is different from the first pharmaceutically acceptable disintegrant (i.e. said second pharmaceutically acceptable disintegrant differs from the first pharmaceutically acceptable disintegrant). According to the invention, the amount of the first pharmaceutically acceptable disintegrant may vary between 0.5% to 15% w/w relative to the total weight of the orodispersible tablet, preferably in an amount of from 2.5% to 10% w/w relative to the total weight of the orodispersible tablet, more preferably in an amount of from 2.5% to 7.5% w/w relative to the total weight of the orodispersible tablet, even more preferably in an amount of from 5% to 7.5% w/w relative to the total weight of the orodispersible tablet. The orodispersible tablet of the invention may comprise the first pharmaceutically acceptable disintegrant in 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0% w/w relative to the total weight of the orodispersible tablet. According to the invention, the amount of the second pharmaceutically acceptable disintegrant may vary between 0.5% to 15% w/w relative to the total weight of the orodispersible tablet, preferably in an amount of from 2.5% to 10% w/w relative to the total weight of the orodispersible tablet, more preferably in an amount of from 2.5% to 7.5% w/w relative to the total weight of the orodispersible tablet, even more preferably in an amount of from 5% to 7.5% w/w relative to the total weight of the orodispersible tablet. The orodispersible tablet of the invention may comprise the second pharmaceutically acceptable disintegrant in 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0% w/w relative to the total weight of the orodispersible tablet.

According to the invention, the total amount of pharmaceutically acceptable disintegrant present in the orodispersible tablet of the invention (i.e. either a single pharmaceutically acceptable disintegrant or the sum of the amount of the first pharmaceutically acceptable disintegrant and the second pharmaceutically acceptable disintegrant which is different from the first pharmaceutically acceptable disintegrant) may range between 1% to 30.0% w/w relative to the total weight of the orodispersible tablet, preferably between 2.5% to 20.0% w/w relative to the total weight of the orodispersible tablet, more preferably between 5.0% to 15.0% w/w relative to the total weight of the orodispersible tablet. The orodispersible tablet of the invention may comprise a total amount of pharmaceutically acceptable disintegrant in 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20.5%, 21.0%, 21.5%, 22.0%, 22.5%, 23.0%, 23.5%, 24.0%, 24.5%, 25.0%, 25.5%, 26.0%, 26.5%, 27.0%, 27.5%, 28.0%, 28.5%, 29.0%, 29.5%, 30.0% w/w relative to the total weight of the orodispersible tablet.

According to the invention, the weight-to-weight ratio of rivaroxaban particles to the total amount of disintegrant present in the orodispersible tablet may range between 1:3.6 to 1:0.12, preferably between 1:2.4 to 1:0.3, more preferably between 1:1.8 to 1:0.6, even more preferably between 1:1.2 to 1:0.9. The orodispersible tablet of the invention may comprise a weight-to-weight ratio of rivaroxaban particles to the total amount of disintegrant being 1:3.6; 1:3.5; 1:3.4; 1:3.3; 1:3.2; 1:3.1; 1:3.0; 1:2.9; 1:2.8; 1:2.7; 1:2.6; 1:2.5; 1:2.4; 1:2.3; 1:2.2; 1:2.1; 1:2.0; 1:1.9; 1:1.8; 1:1.7; 1:1.6; 1:1.5; 1:1.4; 1:1.3; 1:1.2; 1:1.1; 1:1.0; 1:0.9; 1:0.8; 1:0.7; 1:0.6; 1:0.5; 1:0.4; 1:0.3; 1:0.2; 1:0.12.

According to the invention, any pharmaceutically acceptable disintegrant present in the orodispersible tablet (i.e. either a single pharmaceutically acceptable disintegrant or a first pharmaceutically acceptable disintegrant and a second pharmaceutically acceptable disintegrant which is different from the first pharmaceutically acceptable disintegrant) is selected from the list consisting of: crospovidone, croscarmellose sodium, carmellose calcium, carmellose, calcium silicate and sodium starch glycolate. Preferably, the first pharmaceutically acceptable disintegrant and the second pharmaceutically acceptable disintegrant are crospovidone and croscarmellose sodium, respectively.

According to the invention, the orodispersible tablet of the invention may comprise a binder in an amount of from 0.25% to 5.0% w/w relative to the total weight of the orodispersible tablet, more preferably in an amount of from 0.5% to 4.5% w/w relative to the total weight of the orodispersible tablet, even more preferably in an amount of from 1.0% to 3.0% w/w relative to the total weight of the orodispersible tablet. The orodispersible tablet of the invention may comprise a binder in 0.25%, 0.5%, 1.0%, 1.5%, 2.0%, 2.5%, 3.0%, 3.5%, 4.0%, 4.5%, 5.0% w/w relative to the total weight of the orodispersible tablet. The binder may be selected from the group consisting of: hydroxypropyl methyl cellulose, hydroxypropyl cellulose, hydroxyethyl methyl cellulose, hydroxyethyl cellulose, methyl cellulose, ethyl cellulose, sodium carboxymethyl cellulose, preferably being selected from hydroxypropyl methyl cellulose and hydroxypropyl cellulose. The binder is preferably selected from hydroxypropyl methyl cellulose and hydroxypropyl cellulose.

According to the invention, the orodispersible tablet of the invention may comprise a surfactant, which may be selected from the group consisting of sodium lauryl sulfate, polyethylene glycol sorbitan monolaurate, polyethylene glycol sorbitan monopalmitate, polyethylene glycol sorbitan monostearate, polyethylene glycol sorbitan monooleate, polaxamer, sodium dioctyl sulphosuccinate, glycerol monostearate, sorbitan monolaurate. The surfactant is preferably sodium lauryl sulfate.

According to the invention, the orodispersible tablet of the invention may comprise a glidant, which may be selected from the group consisting of colloidal silicon dioxide, hydrophobic colloidal silica, magnesium oxide, magnesium silicate, magnesium trisilicate. The glidant is preferably colloidal silicon dioxide.

According to the invention, the orodispersible tablet of the invention may comprise one or more diluents, which may be selected from the group consisting of mannitol, maltol, sorbitol, maltitol, xylitol, isomalt, erythritol, lactose, starch and its derivatives such as pregelatinized starch, cellulose and its derivatives, in particular microcrystalline cellulose, and calcium phosphate. The most preferred diluents are mannitol and microcrystalline cellulose. Even more preferably, mannitol has a particle size distribution wherein 45% of particles is lower than 150 μm.

According to the invention, the orodispersible tablet of the invention may comprise rivaroxaban particles with a particle size distribution (PSD) such that D(v, 90) is more than 5.00 μm and less than or equal to 20.00 μm, preferably is more than 5.00 μm and less than or equal to 15.00 μm, more preferably is more than 6.00 μm and less than or equal to 10.00 μm; and/or D(v, 50) is more than 2.50 μm and less than or equal to 5.75 μm, preferably is more than 2.50 μm and less than or equal to 5.00 μm, more preferably is more than 2.75 μm and less than or equal to 4.50 μm; and/or D(v, 10) is more than 1.00 μm and less than or equal to 2.00 μm, preferably is more than 1.25 μm and less than or equal to 1.75 μm, more preferably is more than 1.30 μm and less than or equal to 1.70 μm.

In an embodiment, the orodispersible tablet of the invention comprises rivaroxaban particles having a D(v, 90) less than or equal to 20.00 μm, a pharmaceutically acceptable disintegrant, and one or more further pharmaceutically acceptable excipients, wherein said orodispersible tablet is obtainable by a direct compression process of a dry powdered mixture comprising said rivaroxaban particles, the pharmaceutically acceptable disintegrant, and the one or more further pharmaceutically acceptable excipients.

In an embodiment, the orodispersible tablet of the invention comprises rivaroxaban particles having a D(v, 90) less than or equal to 20.00 μm, a pharmaceutically acceptable disintegrant, and one or more further pharmaceutically acceptable excipients, wherein said orodispersible tablet is obtainable by a direct compression process of a dry powdered mixture comprising the steps of: (i) mixing said rivaroxaban particles with the pharmaceutically acceptable disintegrant, and the one or more further pharmaceutically acceptable excipients, (ii) blending the mixture of step (i), (iii) adding one or more lubricants to the mixture of step (ii), (iv) blending the mixture of step (iii), (v) compressing the dry powdered mixture of step (iv) to form the orodispersible tablet.

In an embodiment, the orodispersible tablet of the invention comprises rivaroxaban particles having a D(v, 90) less than or equal to 20.00 μm, a pharmaceutically acceptable disintegrant in an amount of from 0.5% to 15% w/w relative to the total weight of the orodispersible tablet, and one or more further pharmaceutically acceptable excipients, wherein said orodispersible tablet is obtainable by a direct compression process of a dry powdered mixture comprising said rivaroxaban particles, the pharmaceutically acceptable disintegrant, and the one or more further pharmaceutically acceptable excipients.

In an embodiment, the orodispersible tablet of the invention comprises rivaroxaban particles having a D(v, 90) more than 5.00 μm and less than or equal to 20.00 μm, preferably more than 5.00 μm and less than or equal to 15.00 μm, more preferably more than 6.00 μm and less than or equal to 10.00 μm; and/or D(v, 50) more than 2.50 μm and less than or equal to 5.75 μm, preferably more than 2.50 μm and less than or equal to 5.00 μm, more preferably more than 2.75 μm and less than or equal to 4.50 μm; and/or D(v, 10) more than 1.00 μm and less than or equal to 2.00 μm, preferably more than 1.25 μm and less than or equal to 1.75 μm, more preferably more than 1.30 μm and less than or equal to 1.70 μm, a pharmaceutically acceptable disintegrant in an amount of from 0.5% to 15% w/w relative to the total weight of the orodispersible tablet, and one or more further pharmaceutically acceptable excipients, wherein said orodispersible tablet is obtainable by a direct compression process of a dry powdered mixture comprising said rivaroxaban particles, the pharmaceutically acceptable disintegrant, and the one or more further pharmaceutically acceptable excipients. In an embodiment, the orodispersible tablet of the invention comprises rivaroxaban particles having a D(v, 90) less than or equal to 20.00 μm, a pharmaceutically acceptable disintegrant in an amount of from 0.5% to 15% w/w relative to the total weight of the orodispersible tablet, and one or more further pharmaceutically acceptable excipients, wherein the weight-to-weight ratio of rivaroxaban particles to the total amount of disintegrant present in the orodispersible tablet is of from 1:3.6 to 1:0.12, and wherein said orodispersible tablet is obtainable by a direct compression process of a dry powdered mixture comprising said rivaroxaban particles, the pharmaceutically acceptable disintegrant, and the one or more further pharmaceutically acceptable excipients.

In an embodiment, the orodispersible tablet of the invention comprises rivaroxaban particles having a D(v, 90) less than or equal to 20.00 μm, a pharmaceutically acceptable disintegrant in an amount of from 0.5% to 15% w/w relative to the total weight of the orodispersible tablet, a binder in an amount of from 0.25% to 5% w/w relative to the total weight of the orodispersible tablet, and one or more further pharmaceutically acceptable excipients, wherein the weight-to-weight ratio of rivaroxaban particles to the total amount of disintegrant present in the orodispersible tablet is of from 1:3.6 to 1:0.12, and wherein said orodispersible tablet is obtainable by a direct compression process of a dry powdered mixture comprising said rivaroxaban particles, the pharmaceutically acceptable disintegrant, the binder, and the one or more further pharmaceutically acceptable excipients.

In an embodiment, the orodispersible tablet of the invention comprises rivaroxaban particles having a D(v, 90) less than or equal to 20.00 μm, a pharmaceutically acceptable disintegrant in an amount of from 0.5% to 15% w/w relative to the total weight of the orodispersible tablet, a binder in an amount of from 0.25% to 5% w/w relative to the total weight of the orodispersible tablet, a glidant, a diluent, and one or more further pharmaceutically acceptable excipients, wherein the weight-to-weight ratio of rivaroxaban particles to the total amount of disintegrant present in the orodispersible tablet is of from 1:3.6 to 1:0.12, and wherein said orodispersible tablet is obtainable by a direct compression process of a dry powdered mixture comprising said rivaroxaban particles, the pharmaceutically acceptable disintegrant, the binder, the glidant, the diluent, and the one or more further pharmaceutically acceptable excipients.

In an embodiment, the orodispersible tablet of the invention comprises rivaroxaban particles having a D(v, 90) less than or equal to 20.00 μm, a first pharmaceutically acceptable disintegrant and a second pharmaceutically acceptable disintegrant which is different from the first pharmaceutically acceptable disintegrant, and one or more further pharmaceutically acceptable excipients, wherein said orodispersible tablet is obtainable by a direct compression process of a dry powdered mixture comprising said rivaroxaban particles, the first pharmaceutically acceptable disintegrant and the second pharmaceutically acceptable disintegrant which is different from the first pharmaceutically acceptable disintegrant, and the one or more further pharmaceutically acceptable excipients.

In an embodiment, the orodispersible tablet of the invention comprises rivaroxaban particles having a D(v, 90) less than or equal to 20.00 μm, a first pharmaceutically acceptable disintegrant in an amount of from 0.5% to 15% w/w relative to the total weight of the orodispersible tablet, a second pharmaceutically acceptable disintegrant in an amount of from 0.5% to 15% w/w relative to the total weight of the orodispersible tablet, which is different from the first pharmaceutically acceptable disintegrant, and one or more further pharmaceutically acceptable excipients, wherein said orodispersible tablet is obtainable by a direct compression process of a dry powdered mixture comprising said rivaroxaban particles, the first pharmaceutically acceptable disintegrant and the second pharmaceutically acceptable disintegrant which is different from the first pharmaceutically acceptable disintegrant, and the one or more further pharmaceutically acceptable excipients.

In an embodiment, the orodispersible tablet of the invention comprises rivaroxaban particles having a D(v, 90) less than or equal to 20.00 μm, a first pharmaceutically acceptable disintegrant and a second pharmaceutically acceptable disintegrant which is different from the first pharmaceutically acceptable disintegrant, and one or more further pharmaceutically acceptable excipients, wherein the weight-to-weight ratio of rivaroxaban particles to the total amount of disintegrant present in the orodispersible tablet is of from 1:3.6 to 1:0.12, and wherein said orodispersible tablet is obtainable by a direct compression process of a dry powdered mixture comprising said rivaroxaban particles, the first pharmaceutically acceptable disintegrant and the second pharmaceutically acceptable disintegrant which is different from the first pharmaceutically acceptable disintegrant, and the one or more further pharmaceutically acceptable excipients.

In an embodiment, the orodispersible tablet of the invention comprises rivaroxaban particles having a D(v, 90) less than or equal to 20.00 μm, a first pharmaceutically acceptable disintegrant and a second pharmaceutically acceptable disintegrant which is different from the first pharmaceutically acceptable disintegrant, and one or more further pharmaceutically acceptable excipients, wherein the weight-to-weight ratio of rivaroxaban particles to the total amount of disintegrant present in the orodispersible tablet is of from 1:3.6 to 1:0.12, wherein both the first and the second pharmaceutically acceptable disintegrants are selected from the list consisting of: crospovidone, croscarmellose sodium, carmellose calcium, carmellose, calcium silicate and sodium starch glycolate, and wherein said orodispersible tablet is obtainable by a direct compression process of a dry powdered mixture comprising said rivaroxaban particles, the first pharmaceutically acceptable disintegrant and the second pharmaceutically acceptable disintegrant which is different from the first pharmaceutically acceptable disintegrant, and the one or more further pharmaceutically acceptable excipients.

In a preferred embodiment, the orodispersible tablet of the invention comprises rivaroxaban particles having a D(v, 90) less than or equal to 20.00 μm, sodium croscarmellose as a first pharmaceutically acceptable disintegrant, crospovidone as a second pharmaceutically acceptable disintegrant, and one or more further pharmaceutically acceptable excipients, wherein said orodispersible tablet is obtainable by a direct compression process of a dry powdered mixture comprising said rivaroxaban particles, the sodium croscarmellose, the crospovidone and the one or more further pharmaceutically acceptable excipients.

In a preferred embodiment, the orodispersible tablet of the invention comprises rivaroxaban particles having a D(v, 90) less than or equal to 20.00 μm, sodium croscarmellose as a first pharmaceutically acceptable disintegrant in an amount of from 0.5% to 15% w/w relative to the total weight of the orodispersible tablet, crospovidone as a second pharmaceutically acceptable disintegrant in an amount of from 0.5% to 15% w/w relative to the total weight of the orodispersible tablet, and one or more further pharmaceutically acceptable excipients, wherein said orodispersible tablet is obtainable by a direct compression process of a dry powdered mixture comprising said rivaroxaban particles, the sodium croscarmellose, the crospovidone and the one or more further pharmaceutically acceptable excipients.

In a preferred embodiment, the orodispersible tablet of the invention comprises rivaroxaban particles having a D(v, 90) less than or equal to 20.00 μm, sodium croscarmellose as a first pharmaceutically acceptable disintegrant, crospovidone as a second pharmaceutically acceptable disintegrant, and one or more further pharmaceutically acceptable excipients, wherein the weight-to-weight ratio of rivaroxaban particles to the total amount of disintegrant (i.e. the sum of sodium croscarmellose and crospovidone) present in the orodispersible tablet is of from 1:3.6 to 1:0.12, wherein said orodispersible tablet is obtainable by a direct compression process of a dry powdered mixture comprising said rivaroxaban particles, the sodium croscarmellose, the crospovidone and the one or more further pharmaceutically acceptable excipients.

In an embodiment, the orodispersible tablet of the invention comprises rivaroxaban particles having a D(v, 90) less than or equal to 20.00 μm, a first pharmaceutically acceptable disintegrant in an amount of from 0.5% to 15% w/w relative to the total weight of the orodispersible tablet, a second pharmaceutically acceptable disintegrant in an amount of from 0.5% to 15% w/w relative to the total weight of the orodispersible tablet, which is different from the first pharmaceutically acceptable disintegrant, and one or more further pharmaceutically acceptable excipients, wherein the weight-to-weight ratio of rivaroxaban particles to the total amount of disintegrant present in the orodispersible tablet is of from 1:3.6 to 1:0.12, wherein said orodispersible tablet is obtainable by a direct compression process of a dry powdered mixture comprising said rivaroxaban particles, the first pharmaceutically acceptable disintegrant and the second pharmaceutically acceptable disintegrant which is different from the first pharmaceutically acceptable disintegrant, and the one or more further pharmaceutically acceptable excipients.

In an embodiment, the orodispersible tablet of the invention comprises rivaroxaban particles having a D(v, 90) more than 5.00 μm and less than or equal to 20.00 μm, preferably more than 5.00 μm and less than or equal to 15.00 μm, more preferably more than 6.00 μm and less than or equal to 10.00 μm; and/or D(v, 50) more than 2.50 μm and less than or equal to 5.75 μm, preferably more than 2.50 μm and less than or equal to 5.00 μm, more preferably more than 2.75 μm and less than or equal to 4.50 μm; and/or D(v, 10) more than 1.00 μm and less than or equal to 2.00 μm, preferably more than 1.25 μm and less than or equal to 1.75 μm, more preferably more than 1.30 μm and less than or equal to 1.70 μm, a first pharmaceutically acceptable disintegrant in an amount of from 0.5% to 15% w/w relative to the total weight of the orodispersible tablet, a second pharmaceutically acceptable disintegrant in an amount of from 0.5% to 15% w/w relative to the total weight of the orodispersible tablet, which is different from the first pharmaceutically acceptable disintegrant, and one or more further pharmaceutically acceptable excipients, wherein the weight-to-weight ratio of rivaroxaban particles to the total amount of disintegrant present in the orodispersible tablet is of from 1:3.6 to 1:0.12, wherein said orodispersible tablet is obtainable by a direct compression process of a dry powdered mixture comprising said rivaroxaban particles, the first pharmaceutically acceptable disintegrant and the second pharmaceutically acceptable disintegrant which is different from the first pharmaceutically acceptable disintegrant, and the one or more further pharmaceutically acceptable excipients.

In an embodiment, the orodispersible tablet of the invention comprises rivaroxaban particles having a D(v, 90) less than or equal to 20.00 μm, a first pharmaceutically acceptable disintegrant in an amount of from 0.5% to 15% w/w relative to the total weight of the orodispersible tablet, a second pharmaceutically acceptable disintegrant in an amount of from 0.5% to 15% w/w relative to the total weight of the orodispersible tablet, which is different from the first pharmaceutically acceptable disintegrant, a binder in an amount of from 0.25% to 5% w/w relative to the total weight of the orodispersible tablet, and one or more further pharmaceutically acceptable excipients, wherein the weight-to-weight ratio of rivaroxaban particles to the total amount of disintegrant present in the orodispersible tablet is of from 1:3.6 to 1:0.12, wherein said orodispersible tablet is obtainable by a direct compression process of a dry powdered mixture comprising said rivaroxaban particles, the first pharmaceutically acceptable disintegrant and the second pharmaceutically acceptable disintegrant which is different from the first pharmaceutically acceptable disintegrant, the binder, and the one or more further pharmaceutically acceptable excipients.

In an embodiment, the orodispersible tablet of the invention comprises rivaroxaban particles having a D(v, 90) less than or equal to 20.00 μm, a first pharmaceutically acceptable disintegrant in an amount of from 0.5% to 15% w/w relative to the total weight of the orodispersible tablet, a second pharmaceutically acceptable disintegrant in an amount of from 0.5% to 15% w/w relative to the total weight of the orodispersible tablet, which is different from the first pharmaceutically acceptable disintegrant, a binder in an amount of from 0.25% to 5% w/w relative to the total weight of the orodispersible tablet, a glidant, a diluent, and one or more further pharmaceutically acceptable excipients, wherein the weight-to-weight ratio of rivaroxaban particles to the total amount of disintegrant present in the orodispersible tablet is of from 1:3.6 to 1:0.12, wherein said orodispersible tablet is obtainable by a direct compression process of a dry powdered mixture comprising said rivaroxaban particles, the first pharmaceutically acceptable disintegrant and the second pharmaceutically acceptable disintegrant which is different from the first pharmaceutically acceptable disintegrant, the binder, the glidant, the diluent, and the one or more further pharmaceutically acceptable excipients.

As it is mentioned above, a second aspect of the invention relates to a direct compression process for the preparation of the orodispersible tablet of the first aspect of the invention, which comprises the steps of: (i) mixing rivaroxaban particles with the pharmaceutically acceptable disintegrant, and the one or more further pharmaceutically acceptable excipients; (ii) blending the mixture of step (i); (iii) adding one or more lubricants to the mixture of step (ii); (iv) blending the mixture of step (iii); (v) compressing the dry powdered mixture of step (iv) to form the orodispersible tablet.

As mentioned above, the orodispersible tablet of the invention may comprise a filler and a glidant as further pharmaceutically acceptable excipients. In these embodiments, the process for the preparation of the orodispersible tablet of the invention may comprise a first pre-blending step consisting of mixing half amount of the filler present in the orodispersible tablet together with the glidant. That mixture is blended, followed by step (i) mixing rivaroxaban particles with the pharmaceutically acceptable disintegrant, the remaining half amount of the filler present in the orodispersible tablet and the one or more further pharmaceutically acceptable excipients with the first pre-blend; (ii) blending the mixture of step (i); (iii) adding one or more lubricants to the mixture of step (ii); (iv) blending the mixture of step (iii); (v) compressing the dry powdered mixture of step (iv) to form the orodispersible tablet.

In a particular embodiment, the process for the preparation of the orodispersible tablet of the invention comprises a first pre-blending step consisting of mixing half amount of mannitol present in the orodispersible tablet together with colloidal silicone dioxide. That mixture is blended, followed by step (i) mixing rivaroxaban particles with the pharmaceutically acceptable disintegrant, the remaining half amount of the mannitol present in the orodispersible tablet and the one or more further pharmaceutically acceptable excipients with the first pre-blend; (ii) blending the mixture of step (i); (iii) adding one or more lubricants to the mixture of step (ii); (iv) blending the mixture of step (iii); (v) compressing the dry powdered mixture of step (iv) to form the orodispersible tablet.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Reference signs related to drawings and placed in parentheses in a claim, are solely for attempting to increase the intelligibility of the claim and shall not be construed as limiting the scope of the claim. Furthermore, the present invention covers all possible combinations of particular and preferred embodiments described herein.

### EXAMPLES

### Example 1: General Manufacturing Process of Orodispersible Tablets of the Invention

Rivaroxaban particles having a D(v, 90) less than or equal to 20.00 μm, a pharmaceutically acceptable disintegrant, and one or more further pharmaceutically acceptable excipients are weighed and sieved through a 1 mm mesh and further added to a suitable bin blender (Servo-lift bowl). The following further excipients may be optionally added to the mixture present in the bin blender: a second pharmaceutically acceptable disintegrant which is different from the first pharmaceutically acceptable disintegrant, a diluent, a glidant, a binder, a surfactant, a sweetener, a colorant and a flavouring agent. The mixture is blended between 10 and 25 minutes at 34 rpm. Then, a lubricant is weighed and sieved through a 1 mm mesh and further added to the blended mixture. The obtained mixture is blended 5 minutes at 34 rpm. The obtained dry powdered mixture is compressed in a rotatory press into the desired orodispersible tablets of the invention. A general composition is provided below in Table 1.

TABLE 1
general composition of the orodispersible tablets of the invention

| Component | % w/w |
|---|---|
| Rivaroxaban particles having a D(v, 90) | 5-15% |
| less than or equal to 20.00 μm | |
| (First) pharmaceutically acceptable Disintegrant | 0.5-15% |
| Further pharmaceutically acceptable excipients: | |
| Second pharmaceutically acceptable Disintegrant | 0.5-15% |
| Diluent | 60-80% |
| Glidant | 0.15-0.75% |
| Binder | 0.25-5% |
| Surfactant | 0.25-5% |
| Sweetener | 0.50-1% |
| Colorant | 0.01-0.50% |
| Flavour agent | 3-7% |
| Lubricant | 0.50-2.50% |

### Example 2: Orodispersible Tablets Compositions According to the Invention

Formulations 1 to 4-as representative examples according to the invention-are shown in Table 2. Said Formulations 1 to 4 were similarly prepared according to the manufacturing process described in Example 1. Orodispersible tablets of Formulations 1 to 4 comprised rivaroxaban particles having a D(v, 90) less than or equal to 20.00 μm (as measured by using techniques available in the art, such as laser light scattering technique (e.g. by using a Malvern apparatus).

TABLE 2
Composition of Formulations 1 to 4.

| | Formulation 1 | | Formulation 2 | | Formulation 3 | | Formulation 4 | |
|---|---|---|---|---|---|---|---|---|
| Component | % w/w | mg | % w/w | mg | % w/w | mg | % w/w | mg |
| Rivaroxaban | 8.33 | 15.00 | 8.33 | 15.00 | 8.33 | 15.00 | 8.33 | 15.00 |
| D-mannitol | 72.87 | 131.16 | 73.87 | 132.96 | 72.87 | 131.16 | 72.87 | 131.16 |
| Microcrystalline | 5.00 | 9.00 | 5.00 | 9.00 | — | — | 5.00 | 9.00 |
| cellulose | | | | | | | | |
| Lactose | — | — | — | — | 5.00 | 9.00 | — | — |
| monohydrate | | | | | | | | |
| HPMC | 2.00 | 3.60 | 1.00 | 1.80 | 2.00 | 3.60 | 2.00 | 3.60 |
| Sodium | 5.00 | 9.00 | 6.00 | 10.80 | 5.00 | 9.00 | 3.00 | 5.40 |
| croscarmellose | | | | | | | | |
| Crospovidone | 5.00 | 9.00 | 4.00 | 7.20 | 5.00 | 9.00 | — | — |
| Sodium starch | — | — | — | — | — | — | 7.00 | 12.60 |
| glycolate | | | | | | | | |
| Sodium lauryl | 0.60 | 1.08 | 0.60 | 1.08 | 0.60 | 1.08 | 0.60 | 1.08 |
| sulfate | | | | | | | | |
| Colloidal silicone | 0.20 | 0.36 | 0.20 | 0.36 | 0.20 | 0.36 | 0.20 | 0.36 |
| dioxide | | | | | | | | |
| Sodium stearyl | 1.00 | 1.80 | 1.00 | 1.80 | — | — | 1.00 | 1.80 |
| fumarate | | | | | | | | |
| Magnesium | — | — | — | — | 1.00 | 1.80 | — | — |
| stearate | | | | | | | | |
| Total | 100.00 | 180.00 | 100.00 | 180.00 | 100.00 | 180.00 | 100.00 | 180.00 |

### Example 3. Characterization of Orodispersible Tablets According to the Invention: Disintegration Test, Hardness, Thickness, Friability Test

Formulations 1 to 4 (according to the invention) were tested for disintegration time following Test A of the European Pharmacopeia (edition 10.0, page 323), water having pH=7, at 37° C. and 30 cycles per minute. The results are shown in Table 3.

Formulations 1 to 4 (according to the invention) were also submitted for friability test in accordance with the guidelines of the European Pharmacopeia, edition 10.0, pp. 336-337. The results are shown in Table 3. Furthermore, Formulations 1 to 4 were also characterized by their hardness and thickness.

TABLE 3
Characterization of orodispersible tablets according to the invention

| Orodispersible | Disintegration | Friability | Hardness | Thickness |
|---|---|---|---|---|
| tablet | time (sec.) | (%) | (N) | (mm) |
| Formulation 1 | 38 | 0.27 | 36 | 3.83 |
| Formulation 2 | 20 | 0.23 | 130 | 3.40 |
| Formulation 3 | 20 | 0.44 | 92 | 3.31 |
| Formulation 4 | 34 | 0.12 | 149 | 3.28 |

As shown in Table 3, all orodispersible tablets of Formulations 1 to 4 (according to the invention) complied with the requirement of being orodispersible tablets, in accordance with the definition given by the European Pharmacopeia, edition 10.0, page 939. Furthermore, all disintegrated in around 60 seconds or less—thus being considered as fast/quickly orally disintegrating tablets.

Moreover, all orodispersible tablets of Formulations 1 to 4 (according to the invention) complied with the requirement of having a friability of less than 1% in accordance with guidelines of the European Pharmacopeia.

### Example 4. Dissolution Profile of Orodispersible Tablets According to the Invention

Dissolution profiles of Formulations 1 to 4 (according to the invention) and dissolution profile of a commercially available film-coated tablet of Xarelto® (dosage of 15 mg) were measured according to the following dissolution method: pH=4.5 with the presence of 0.4% sodium lauryl sulfate, using Apparatus II (paddle), placing the orodispersible tablet in 900 mL at 37° C., stirring at 75 revolutions per minute and N=6. The results are shown in Tables 4-5.

TABLE 4
Dissolution profiles of Xarelto ® film-
coated tablet and Formulations 1-2.

| | Xarelto ® (FCT) | | Formulation 1 | | Formulation 2 | |
|---|---|---|---|---|---|---|
| T | Dis. | RSD | Dis. | RSD | Dis. | RSD |
| (min) | (%) | (%) | (%) | (%) | (%) | (%) |
| 0 | 0.0 | 0.0 | 0.0 | 0.0 | 0.0 | 0.0 |
| 5 | 49.2 | 16.1 | 66.8 | 3.9 | 61.4 | 33.1 |
| 10 | 90.7 | 5.2 | 85.0 | 5.2 | 82.2 | 4.3 |
| 15 | 99.4 | 1.5 | 89.7 | 3.6 | 86.0 | 1.8 |
| 20 | 100.8 | 1.0 | 92.6 | 3.1 | 90.1 | 1.2 |
| 30 | 101.2 | 1.0 | 95.9 | 2.7 | 93.5 | 2.3 |
| 45 | 101.5 | 1.1 | 97.8 | 3.3 | 94.8 | 2.5 |
| 60 | 101.7 | 1.1 | 98.7 | 3.9 | 95.3 | 3.7 |

TABLE 5
Dissolution profiles of Formulations 3-4.

| | |
|---|---|
| Formulation 3 | Formulation 4 |

| T | Dis. | RSD | Dis. | RSD |
|---|---|---|---|---|
| (min) | (%) | (%) | (%) | (%) |
| 0 | 0.0 | 0.0 | 0.0 | 0.0 |
| 5 | 51.9 | 11.3 | 53.9 | 3.8 |
| 10 | 82.3 | 2.0 | 70.7 | 2.6 |
| 15 | 90.0 | 1.3 | 79.3 | 1.9 |
| 20 | 93.5 | 1.4 | 85.1 | 2.0 |
| 30 | 96.3 | 1.6 | 91.1 | 2.0 |
| 45 | 97.5 | 1.6 | 94.8 | 1.6 |
| 60 | 97.6 | 1.6 | 96.3 | 1.7 |

By having such dissolution profile, and thus being sufficiently equivalent to currently marketed Xarelto® conventional film-coated tablets, rivaroxaban orodispersible tablets of the invention avoids the risk of being either suprabioequivalent or infrabioequivalent, which may affect the toxicity and/or the efficacy of said orodispersible tablet.

### CITATION LIST

WO05060940
JP2021195335
European Pharmacopeia, edition 10.0, page 939
European Pharmacopeia, edition 10.0, page 323
European Pharmacopeia, edition 10.0, pp. 336-337
Guideline for Bioequivalence (CPMP/EWP/QWP/1401/98 Rev.1/Corr., 2010)

1. An orodispersible tablet comprising rivaroxaban particles having a D(v, 90) less than or equal to 20.00 μm, a pharmaceutically acceptable disintegrant, and one or more further pharmaceutically acceptable excipients,
wherein said orodispersible tablet is obtainable by a direct compression process of a dry powdered mixture comprising said rivaroxaban particles, the pharmaceutically acceptable disintegrant, and the one or more further pharmaceutically acceptable excipients.

2. The orodispersible tablet according to claim 1, wherein said orodispersible tablet is disintegrated in less than 3 minutes, preferably in less than 2 minutes, and more preferably in less than 1 minute, wherein the disintegration test was performed using a European Pharmacopeia disintegration apparatus A, placing the dosage form in water having pH=7 at 37° C. and 30 cycles per minute.

3. The orodispersible tablet according to any of claims 1, wherein said orodispersible tablet further comprises a second pharmaceutically acceptable disintegrant which is different from the first pharmaceutically acceptable disintegrant.

4. The orodispersible tablet according to claim 3, wherein said orodispersible tablet comprises the first pharmaceutically acceptable disintegrant in an amount of from 0.5% to 15% w/w relative to the total weight of the orodispersible tablet, preferably in an amount of from 2.5% to 10% w/w relative to the total weight of the orodispersible tablet, more preferably in an amount of from 2.5% to 7.5% w/w relative to the total weight of the orodispersible tablet, even more preferably in an amount of from 5% to 7.5% w/w relative to the total weight of the orodispersible tablet, and
wherein said orodispersible tablet comprises the second pharmaceutically acceptable disintegrant in an amount of from 0.5% to 15% w/w relative to the total weight of the orodispersible tablet, preferably in an amount of from 2.5% to 10% w/w relative to the total weight of the orodispersible tablet, more preferably in an amount of from 2.5% to 7.5% w/w relative to the total weight of the orodispersible tablet, even more preferably in an amount of from 5% to 7.5% w/w relative to the total weight of the orodispersible tablet.

5. The orodispersible tablet according to claim 1, wherein the weight-to-weight ratio of rivaroxaban particles to the total amount of disintegrant present in the orodispersible tablet is of from 1:3.6 to 1:0.12, preferably of from to 1:2.4 to 1:0.3, more preferably of from 1:1.8 to 1:0.6, even more preferably of from 1:1.2 to 1:0.9.

6. The orodispersible tablet according to claim 1, wherein any pharmaceutically acceptable disintegrant present in the orodispersible tablet is selected from the list consisting of: crospovidone, croscarmellose sodium, carmellose calcium, carmellose, calcium silicate and sodium starch glycolate.

7. The orodispersible tablet according to claim 1, wherein said orodispersible tablet comprises a binder in an amount of from 0.25% to 5% w/w relative to the total weight of the orodispersible tablet.

8. The orodispersible tablet according to claim 7, wherein the binder is selected from the group consisting of: hydroxypropyl methyl cellulose, hydroxypropyl cellulose, hydroxy ethyl methyl cellulose, hydroxy ethyl cellulose, methyl cellulose, ethyl cellulose, sodium carboxymethyl cellulose, preferably being selected from hydroxypropyl methyl cellulose and hydroxypropyl cellulose.

9. The orodispersible tablet according to claim 1, wherein said orodispersible tablet comprises a surfactant selected from the group consisting of:
sodium lauryl sulfate, polyethylene glycol sorbitan monolaurate, polyethylene glycol sorbitan monopalmitate, polyethylene glycol sorbitan monostearate, polyethylene glycol sorbitan monooleate, polaxamer, sodium dioctyl sulphosuccinate, glycerol monostearate, sorbitan monolaurate.

10. The orodispersible tablet according to claim 1, wherein said orodispersible tablet comprises a glidant, preferably being colloidal silicon dioxide.

11. The orodispersible tablet according to claim 1, wherein said orodispersible tablet comprises a diluent.

12. The orodispersible tablet according to claim 1, wherein rivaroxaban particles have a particle size distribution (PSD) in which D(v, 90) is more than 5.00 μm and less than or equal to 20.00 μm, preferably is more than 5.00 μm and less than or equal to 15.00 μm, more preferably is more than 6.00 μm and less than or equal to 10.00 μm; and/or
D(v, 50) is more than 2.50 μm and less than or equal to 5.75 μm, preferably is more than 2.50 μm and less than or equal to 5.00 μm, more preferably is more than 2.75 μm and less than or equal to 4.50 μm; and/or
D(v, 10) is more than 1.00 μm and less than or equal to 2.00 μm, preferably is more than 1.25 μm and less than or equal to 1.75 μm, more preferably is more than 1.30 μm and less than or equal to 1.70 μm.

13. The orodispersible tablet according to claim 1, wherein the direct compression process comprises the steps of:
(i) mixing rivaroxaban particles with the pharmaceutically acceptable disintegrant, and the one or more further pharmaceutically acceptable excipients;
(ii) blending the mixture of step (i);
(iii) adding one or more lubricants to the mixture of step (ii);
(iv) blending the mixture of step (iii);
(v) compressing the dry powdered mixture of step (iv) to form the orodispersible tablet.

14. A process for the preparation of the orodispersible tablet according to claim 1, which comprises the steps of:
(i) mixing rivaroxaban particles with the pharmaceutically acceptable disintegrant, and the one or more further pharmaceutically acceptable excipients;
(ii) blending the mixture of step (i);
(iii) adding one or more lubricants to the mixture of step (ii);
(iv) blending the mixture of step (iii);
(v) compressing the dry powdered mixture of step (iv) to form the orodispersible tablet.

15. The orodispersible tablet according to claim 1 for use in the treatment of: a) prevention of venous thromboembolism (VTE) in adult patients undergoing elective hip or knee replacement surgery; and/or b) treatment of deep vein thrombosis (DVT) and pulmonary embolism (PE), and prevention of recurrent DVT and PE in adults; and/or c) prevention of stroke and systemic embolism in adult patients with non-valvular atrial fibrillation with one or more risk factors, such as congestive heart failure, hypertension, age ≥75 years, diabetes mellitus, prior stroke or transient ischaemic attack; and/or d) treatment of venous thromboembolism (VTE) and prevention of VTE recurrence in children and adolescents aged less than 18 years and weighing either from 30 kg to 50 kg or more than 50 kg after at least 5 days of initial parenteral anticoagulation treatment; and/or e) treatment of venous thromboembolism (VTE) and prevention of VTE recurrence in term neonates, infants and toddlers, children, and adolescents aged less than 18 years after at least 5 days of initial parenteral anticoagulation treatment; and/or f) inhibition of ischemic stroke and systemic embolism in patients with non-valvular atrial fibrillation; and/or g) treatment and suppression of recurrence of venous thromboembolism; and/or h) co-administered with acetylsalicylic acid (ASA) alone or with ASA plus clopidogrel or ticlopidine, prevention of atherothrombotic events in adult patients after an acute coronary syndrome (ACS) with elevated cardiac biomarkers;
and/or i) co-administered with acetylsalicylic acid (ASA), prevention of atherothrombotic events in adult patients with coronary artery disease (CAD) or symptomatic peripheral artery disease (PAD) at high risk of ischaemic events.
